(19) United States
(12) Patent Application Publication
(10) Pub. No.: US 2024/0390320 A1
(43) Pub. Date: Nov. 28, 2024

(54) COMPOSITION FOR REDUCING BODY FAT CONTAINING GALLOCATECHIN GALLATE AND ISOQUERCITRIN

(71) Applicant: AMOREPACIFIC CORPORATION, Seoul (KR)

(72) Inventors: Hyun Woo JEONG, Yongin-si (KR); Wanki KIM, Yongin-si (KR); Jong Hwa ROH, Yongin-si (KR)

(73) Assignee: AMOREPACIFIC CORPORATION, Seoul (KR)

(21) Appl. No.: 18/663,736

(22) Filed: May 14, 2024

(30) Foreign Application Priority Data
May 26, 2023 (KR) 10-2023-0068217

(51) Int. Cl. A61K 31/353; A61K 31/7048; A61P 3/04

(52) U.S. Cl. CPC A61K 31/353; A61K 31/7048; A61P 3/04

(57) ABSTRACT

Disclosed herein is a method for inhibiting lipogenesis; inhibiting fat accumulation; promoting fat breakdown; reducing body fat; and/or preventing, improving or treating obesity or metabolic diseases. In one aspect, Gallocatechin Gallate (GCG) and Isoquercitrin (IQC) of the present invention have excellent lipid metabolism control effects, can increase mitochondria in adipocytes, and promote the production of brown fat. Therefore, in one aspect, the present invention can provide a method for preventing, improving, or treating obesity or metabolic diseases.

### CROSS-REFERENCE TO RELATED APPLICATION

The present application claims priority to Korean Patent Application No. 10-2023-0068217, filed May 26, 2023, the entire contents of which are hereby incorporated by this reference.

### BACKGROUND OF THE INVENTION

### Field of the Invention

Disclosed herein is a composition for inhibiting lipogenesis, inhibiting fat accumulation or promoting fat breakdown.

### Description of the Related Art

Obesity is a condition caused by an excessive increase in body fat and metabolic abnormalities, and the number of obese patients has been steadily increasing over the past 20 years. Obesity is considered a major risk factor for various adult diseases such as high blood pressure, diabetes, and cardiovascular disease, as well as cancer. Therefore, anti-obesity treatment is important not only for weight loss but also for improving various diseases such as hyperglycemia, dyslipidemia, and arteriosclerotic heart disease related to obesity.

### SUMMARY OF THE INVENTION

One object of the present invention is to provide a composition for inhibiting lipogenesis; inhibiting fat accumulation; promoting fat breakdown; reducing body fat; and/or preventing, improving or treating obesity or metabolic diseases.

Another object of the present invention is to provide a pharmaceutical composition for preventing or treating obesity or metabolic diseases.

Another object of the present invention is to provide a food composition for inhibiting lipogenesis; inhibiting fat accumulation; promoting fat breakdown; reducing body fat; and/or preventing or improving obesity or metabolic diseases.

In order to achieve the above object, in one aspect, the present invention provides a composition for inhibiting lipogenesis; inhibiting fat accumulation; promoting fat breakdown; reducing body fat; and/or preventing, improving or treating obesity or metabolic diseases, comprising Gallocatechin Gallate (GCG) and Isoquercitrin (IQC) as active ingredients.

In another aspect, the present invention provides a pharmaceutical composition for preventing or treating obesity or metabolic diseases, comprising Gallocatechin Gallate (GCG) and Isoquercitrin (IQC) as active ingredients.

In another aspect, the present invention provides a food composition for inhibiting lipogenesis; inhibiting fat accumulation; promoting fat breakdown; reducing body fat; and/or preventing or improving obesity or metabolic diseases, comprising Gallocatechin Gallate (GCG) and Isoquercitrin (IQC) as active ingredients.

### Advantageous Effects of Invention

In one aspect, the composition of the present invention has excellent lipid metabolism control efficacy and thus exhibits significant effects of inhibiting lipogenesis, inhibiting fat accumulation, or promoting fat breakdown.

In another aspect, the composition of the present invention can increase mitochondria in adipocytes and effectively promote the production of brown fat.

### BRIEF DESCRIPTION OF THE DRAWINGS

FIG. 1 compares changes in the expression of genes related to lipogenesis in adipocytes according to the composition ratio of a mixture (APGQ) of Gallocatechin Gallate (GCG) and Isoquercitrin (IQC).

FIG. 2 compares the increase in the expression of genes related to oxidation of fatty acid by APGQ composition ratio.

FIGS. 3A-3B compare changes in the expression of genes related to fat metabolism by APGQ composition ratio. FIG. 3A relates to genes that induce lipogenesis, and FIG. 3B relates to genes that promote fatty acid oxidation.

FIG. 4 relates to the increase in mitochondrial structural gene expression in adipocytes by APGQ.

FIG. 5 relates to the increase in the number of mitochondria in adipocytes by APGQ.

FIG. 6 relates to the increase in UCP expression in adipocytes by APGQ.

FIG. 7 relates to the increase in intracellular oxygen consumption by APGQ. XF Analyzer (Seahorse) was used to measure intracellular oxygen consumption.

FIG. 8 relates to the inhibitory effect of APGQ on lipid accumulation in adipocytes.

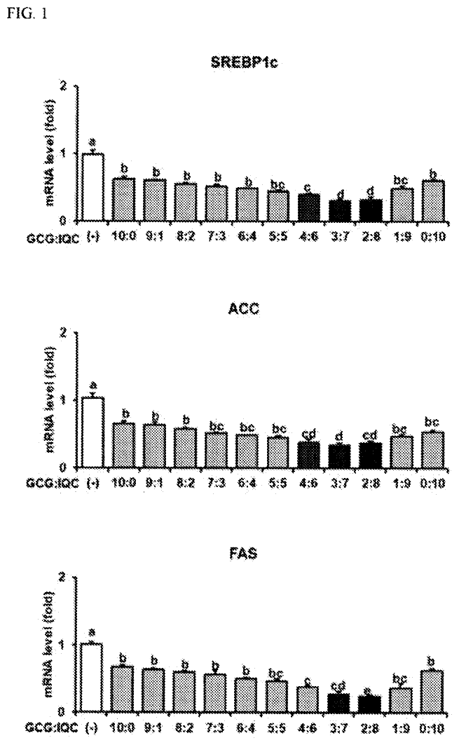

In FIGS. 1 to 8, experimental groups showing statistically significant differences (P<0.01) are indicated with different symbols.

### DETAILED DESCRIPTION OF THE INVENTION

Hereinafter, the present invention will be described in detail.

In one aspect, the present invention provides a composition for inhibiting lipogenesis; inhibiting fat accumulation; promoting fat breakdown; reducing body fat; and/or preventing, improving or treating obesity or metabolic diseases, comprising Gallocatechin Gallate (GCG) and Isoquercitrin (IQC) in a weight ratio of 1:1 to 1:9.

In an exemplary embodiment, the gallocatechin gallate and isoquercitrin may be included in weight ratio of 1:1 or more, 1:1.1 or more, 1:1.2 or more, 1:1.3 or more, 1:1.4 or more, 1:1.5 or more, 1:1.6 or more, 1:1.7 or more, 1:1.8 or more, 1:1.9 or more, 1:2.0 or more, 1:2.1 or more, 1:2.2 or more, 1:2.3 or more, 1:2.4 or more, or 1:2.5 or more, and may be included in weight ratio of 1:9 or less, 1:8 or less, 1:7 or less, 1:6 or less, 1:5 or less, 1:4 or less, 1:3.5 or less, 1:3.4 or less, 1:3.3 or less, 1:3.2 or less, 1:3.1 or less, 1:3 or less, 1:2.9 or less, 1:2.8 or less, 1:2.7 or less, or 1:2.6 or less. When the gallocatechin gallate and isoquercitrin are included in the above weight ratio, the effects of inhibiting lipogenesis, inhibiting fat accumulation or promoting fat breakdown can be effectively exhibited. For example, the gallocatechin gallate and isoquercitrin may be included in a weight ratio of 1:1 to 1:9 or 1:1.5 to 1:4, but are not limited thereto.

In one exemplary embodiment, the composition may be administered to a subject in need of reducing the expression of one or more genes selected from the group consisting of SREBP1c, ACC, and FAS.

In one exemplary embodiment, the composition may be administered to a subject in need of increasing the expression of one or more genes selected from the group consisting of ACO, CPT, PPARα, tfam, NDUFA9, COX4, ATP5a, UCP1, and UCP2.

In one exemplary embodiment, the composition may be administered to a subject in need of increasing mitochondria in adipocytes.

In one exemplary embodiment, the composition may be administered to a subject in need of promoting the production of brown fat.

In one exemplary embodiment, the daily dosage of the active ingredients of the composition may be 5 mg/kg or more, 6 mg/kg or more, 7 mg/kg or more, 8 mg/kg or more, 9 mg/kg or more, 10 mg/kg or more, 20 mg/kg or more, 30 mg/kg or more, 40 mg/kg or more, 50 mg/kg or more, 60 mg/kg or more, 70 mg/kg or more, 80 mg/kg or more, 90 mg/kg or more, 100 mg/kg or more, 1 g/kg or more, 2 g/kg or more, 3 g/kg or more, 4 g/kg or more, 5 g/kg or more, or 6 g/kg or more, and may be 60 g/kg or less, 59 g/kg or less, 58 g/kg or less, 57 g/kg or less, 56 g/kg or less, 55 g/kg or less, 54 g/kg or less, 53 g/kg or less, 52 g/kg or less, or 51 g/kg or less. At the above dosage, the effect of inhibiting lipogenesis, inhibiting fat accumulation, or promoting fat breakdown is excellent. If the dosage is less than the above, the effect of inhibiting lipogenesis, inhibiting fat accumulation, or promoting fat breakdown is minimal, and if the dosage is higher than the above, problems such as toxicity may occur. The above administration may be administered once or in divided doses several times a day. For example, it may be administered 2 to 24 times per day, 1 to 2 times per 3 days, 1 to 6 times per week, 1 to 10 times per 2 weeks, 1 to 15 times per 3 weeks, 1 to 3 times per 4 weeks, or 1 to 12 times per a year, but are not limited thereto.

In another aspect, the present invention provides a method for inhibiting lipogenesis; inhibiting fat accumulation; promoting fat breakdown; reducing body fat; and/or preventing, improving or treating obesity or metabolic diseases, comprising administering an effective amount of the Gallocatechin Gallate (GCG) and Isoquercitrin (IQC) in a weight ratio of 1:1 to 1:9 to subject in need thereof.

In another aspect, the present invention relates a use of Gallocatechin Gallate (GCG) and Isoquercitrin (IQC) for manufacturing a composition for inhibiting lipogenesis; inhibiting fat accumulation; promoting fat breakdown; reducing body fat; and/or preventing, improving or treating obesity or metabolic diseases, and the composition may comprise the gallocatechin gallate and isoquercitrin in a weight ratio of 1:1 to 1:9.

In another aspect, the present invention relates a use of Gallocatechin Gallate (GCG) and Isoquercitrin (IQC) for inhibiting lipogenesis; inhibiting fat accumulation; promoting fat breakdown; reducing body fat; and/or preventing, improving or treating obesity or metabolic diseases, and the weight ratio of the gallocatechin gallate and isoquercitrin may be 1:1 to 1:9.

In another aspect, the present invention relates to a non-therapeutic or therapeutic use of Gallocatechin Gallate (GCG) and Isoquercitrin (IQC).

In another aspect, the present invention relates to a pharmaceutical composition for preventing or treating obesity or metabolic diseases, comprising the Gallocatechin Gallate (GCG) and Isoquercitrin (IQC) in a weight ratio of 1:1 to 1:9.

As used herein, the term “obesity” refers to a condition in which there is excessive adipose tissue in the body. Men are said to be obese when their body fat is more than 25% of their body weight, and women are said to be obese when their body fat is more than 30% of their body weight. Obesity occurs when the calories consumed from food are more than the calories consumed by moving the body. When obesity occurs, the person looks fat and is often accompanied by symptoms such as shortness of breath, joint pain, diabetes, and high blood pressure.

As used herein, the term “prevention” refers to any action that inhibits or delays obesity or metabolic disease by administering the pharmaceutical composition. The term “treatment” refers to any action in which the symptoms of an individual suspected or suffering from obesity or metabolic disease are improved or beneficially changed by administration of the pharmaceutical composition.

In one exemplary embodiment, the metabolic disease is a metabolic disease derived from obesity and may include one or more selected from the group consisting of diabetes, hyperlipidemia, heart disease, stroke, arteriosclerosis, and fatty liver.

In one exemplary embodiment, the gallocatechin gallate and isoquercitrin may be included in weight ratio of 1:1 or more, 1:1.1 or more, 1:1.2 or more, 1:1.3 or more, 1:1.4 or more, 1:1.5 or more, 1:1.6 or more, 1:1.7 or more, 1:1.8 or more, 1:1.9 or more, 1:2.0 or more, 1:2.1 or more, 1:2.2 or more, 1:2.3 or more, 1:2.4 or more, or 1:2.5 or more, and may be included in weight ratio of 1:9 or less, 1:8 or less, 1:7 or less, 1:6 or less, 1:5 or less, 1:4 or less, 1:3.5 or less, 1:3.4 or less, 1:3.3 or less, 1:3.2 or less, 1:3.1 or less, 1:3 or less, 1:2.9 or less, 1:2.8 or less, 1:2.7 or less, or 1:2.6 or less. When the gallocatechin gallate and isoquercitrin are included in the above weight ratio, the effect of preventing or treating obesity or metabolic diseases can be effectively exhibited. For example, the gallocatechin gallate and isoquercitrin may be included in a weight ratio of 1:1 to 1:9 or 1:1.5 to 1:4, but are not limited thereto.

In one exemplary embodiment, the pharmaceutical composition may be administered to a subject in need of reducing the expression of one or more genes selected from the group consisting of SREBP1c, ACC, and FAS.

In one exemplary embodiment, the pharmaceutical composition may be administered to a subject in need of increasing the expression of one or more genes selected from the group consisting of ACO, CPT, PPARα, tfam, NDUFA9, COX4, ATP5a, UCP1, and UCP2.

In one exemplary embodiment, the pharmaceutical composition may be administered to a subject in need of increasing mitochondria in adipocytes.

In one exemplary embodiment, the pharmaceutical composition may be administered to a subject in need of promoting the production of brown fat.

In one exemplary embodiment, the daily dosage of the active ingredients of the pharmaceutical composition may be 5 mg/kg or more, 6 mg/kg or more, 7 mg/kg or more, 8 mg/kg or more, 9 mg/kg or more, 10 mg/kg or more, 20 mg/kg or more, 30 mg/kg or more, 40 mg/kg or more, 50 mg/kg or more, 60 mg/kg or more, 70 mg/kg or more, 80 mg/kg or more, 90 mg/kg or more, 100 mg/kg or more, 1 g/kg or more, 2 g/kg or more, 3 g/kg or more, 4 g/kg or more, 5 g/kg or more, or 6 g/kg or more, and may be 60 g/kg or less, 59 g/kg or less, 58 g/kg or less, 57 g/kg or less, 56 g/kg or less, 55 g/kg or less, 54 g/kg or less, 53 g/kg or less, 52 g/kg or less, or 51 g/kg or less. At the above dosage, the effect of preventing or treating obesity or metabolic diseases is excellent. If the dosage is less than the above, the effect of preventing or treating obesity or metabolic diseases is minimal, and if the dosage is higher than the above, problems such as toxicity may occur. The above administration may be administered once or in divided doses several times a day. For example, it may be administered 2 to 24 times per day, 1 to 2 times per 3 days, 1 to 6 times per week, 1 to 10 times per 2 weeks, 1 to 15 times per 3 weeks, 1 to 3 times per 4 weeks, or 1 to 12 times per a year, but are not limited thereto.

In another aspect, the present invention relates to a use of Gallocatechin Gallate (GCG) and Isoquercitrin (IQC) for manufacturing a pharmaceutical composition for preventing or treating obesity or metabolic diseases, and the pharmaceutical composition may comprise the gallocatechin gallate and isoquercitrin in a weight ratio of 1:1 to 1:9.

In one exemplary embodiment, the pharmaceutical composition may be provided in any formulation suitable for topical administration. For example, it may be administered orally, transdermally, intravenously, intramuscularly, or by subcutaneous injection. As an example, the pharmaceutical composition may be an injection, a solution for external use on the skin, a suspension, an emulsion, a gel, a patch, or a spray, but is not limited thereto. The formulation may be easily manufactured according to conventional methods in the field, and surfactants, excipients, wetting agents, emulsification accelerators, suspending agents, salts or buffers for adjusting osmotic pressure, colorants, flavorings, stabilizers, preservatives, or other commonly used supplements may be used appropriately.

In one exemplary embodiment, the active ingredients of the pharmaceutical composition may vary depending on the subject's age, gender, weight, pathological condition and its severity, route of administration, or the judgment of the prescriber. Determination of the appropriate dosage to use based on these factors is within the level of one skilled in the art.

In another aspect, the present invention relates to a food composition for inhibiting lipogenesis; inhibiting fat accumulation; promoting fat breakdown; reducing body fat; and/or preventing or improving obesity or metabolic diseases, comprising the Gallocatechin Gallate (GCG) and Isoquercitrin (IQC) in a weight ratio of 1:1 to 1:9.

As used herein, the term “improvement” refers to any action in which obesity or metabolic disease is improved or beneficially changed by administration of the composition.

In one exemplary embodiment, the metabolic disease is a metabolic disease derived from obesity and may include one or more selected from the group consisting of diabetes, hyperlipidemia, heart disease, stroke, arteriosclerosis, and fatty liver.

In one exemplary embodiment, the gallocatechin gallate and isoquercitrin may be included in weight ratio of 1:1 or more, 1:1.1 or more, 1:1.2 or more, 1:1.3 or more, 1:1.4 or more, 1:1.5 or more, 1:1.6 or more, 1:1.7 or more, 1:1.8 or more, 1:1.9 or more, 1:2.0 or more, 1:2.1 or more, 1:2.2 or more, 1:2.3 or more, 1:2.4 or more, or 1:2.5 or more, and may be included in weight ratio of 1:9 or less, 1:8 or less, 1:7 or less, 1:6 or less, 1:5 or less, 1:4 or less, 1:3.5 or less, 1:3.4 or less, 1:3.3 or less, 1:3.2 or less, 1:3.1 or less, 1:3 or less, 1:2.9 or less, 1:2.8 or less, 1:2.7 or less, or 1:2.6 or less. When the gallocatechin gallate and isoquercitrin are included in the above weight ratio, the effect of preventing or improving obesity or metabolic diseases can be effectively exhibited. For example, the gallocatechin gallate and isoquercitrin may be included in a weight ratio of 1:1 to 1:9 or 1:1.5 to 1:4, but are not limited thereto.

In one exemplary embodiment, the food composition may be administered to a subject in need of reducing the expression of one or more genes selected from the group consisting of SREBP1c, ACC, and FAS.

In one exemplary embodiment, the food composition may be administered to a subject in need of increasing the expression of one or more genes selected from the group consisting of ACO, CPT, PPARα, tfam, NDUFA9, COX4, ATP5a, UCP1, and UCP2.

In one exemplary embodiment, the food composition may be administered to a subject in need of increasing mitochondria in adipocytes.

In one exemplary embodiment, the food composition may be administered to a subject in need of promoting the production of brown fat.

In one exemplary embodiment, the daily dosage of the food composition may be 5 mg/kg or more, 6 mg/kg or more, 7 mg/kg or more, 8 mg/kg or more, 9 mg/kg or more, 10 mg/kg or more, 20 mg/kg or more, 30 mg/kg or more, 40 mg/kg or more, 50 mg/kg or more, 60 mg/kg or more, 70 mg/kg or more, 80 mg/kg or more, 90 mg/kg or more, 100 mg/kg or more, 1 g/kg or more, 2 g/kg or more, 3 g/kg or more, 4 g/kg or more, 5 g/kg or more, or 6 g/kg or more, and may be 60 g/kg or less, 59 g/kg or less, 58 g/kg or less, 57 g/kg or less, 56 g/kg or less, 55 g/kg or less, 54 g/kg or less, 53 g/kg or less, 52 g/kg or less, or 51 g/kg or less. At the above dosage, the effect of preventing or improving obesity or metabolic diseases, or reducing body fat is excellent. If the dosage is less than the above, the effect of preventing or improving obesity or metabolic diseases, or reducing body fat is minimal, and if the dosage is higher than the above, problems such as toxicity may occur. The above administration may be administered once or in divided doses several times a day. For example, it may be administered 2 to 24 times per day, 1 to 2 times per 3 days, 1 to 6 times per week, 1 to 10 times per 2 weeks, 1 to 15 times per 3 weeks, 1 to 3 times per 4 weeks, or 1 to 12 times per a year, but are not limited thereto.

In another aspect, the present invention relates to a use of Gallocatechin Gallate (GCG) and Isoquercitrin (IQC) for manufacturing a food composition for inhibiting lipogenesis; inhibiting fat accumulation; promoting fat breakdown; reducing body fat; and/or preventing or improving obesity or metabolic diseases, and the food composition may comprise the gallocatechin gallate and isoquercitrin in a weight ratio of 1:1 to 1:9.

In one exemplary embodiment, when the composition is used as an additive to a health functional food, it may be added as is or used together with other foods or food ingredients, and may be used appropriately according to conventional methods. The amount of active ingredients mixed may be appropriately determined depending on each purpose of use, such as prevention, health, or treatment. The formulation of health functional foods may be in the form of powders, granules, pills, tablets, capsules, as well as general foods or beverages.

In one exemplary embodiment, there is no particular limitation on the type of the health functional food, and examples of foods to which the composition can be added may be meat, confectionery, noodles, gum, dairy products including ice cream, various soups, beverages, tea, drinks, alcoholic beverages, and vitamin complexes, etc., and may include all foods in the conventional sense.

In one exemplary embodiment, when manufacturing the health functional food or beverage, the composition may be added in an amount of 15 parts by weight or less, preferably 10 parts by weight or less, based on 100 parts by weight of the raw material. However, in the case of long-term intake for the purpose of health and hygiene, or health control, the amount may be below the above range.

In one exemplary embodiment, the beverage among the health functional foods may contain various flavoring agents or natural carbohydrates as additional ingredients like a typical beverage. The above-mentioned natural carbohydrates may be monosaccharides such as glucose and fructose, disaccharides such as maltose and sucrose, polysaccharides such as dextrin and cyclodextrin, and sugar alcohols such as xylitol, sorbitol, and erythritol, etc. As a sweetener, natural sweeteners such as thaumatin and stevia extract or synthetic sweeteners such as saccharin and aspartame may be used. The ratio of the natural carbohydrate may be about 0.01 to 0.04 g, preferably about 0.02 to 0.03 g, per 100 mL of the beverage according to the present invention, but is not limited thereto.

In one exemplary embodiment, in addition to the above, the health functional food according to the present invention may contain various nutrients, vitamins, electrolytes, flavors, colorants, pectic acid and its salts, alginic acid and its salts, organic acids, protective colloidal thickeners, pH conditioners, stabilizers, preservatives, glycerin, alcohol, and carbonating agents used in carbonated beverages. In addition, the health functional food according to the present invention may contain pulp for manufacturing natural fruit juice, fruit juice drinks, and vegetable drinks. These ingredients may be used independently or in combination. The ratio of these additives is not limited, but is generally selected in the range of 0.01 to 0.1 parts by weight based on 100 parts by weight of the health functional food according to the present invention.

Hereinafter, the configuration and effects of the present invention will be described in more detail through examples. However, the examples below are provided only for illustrative purposes to aid understanding of the present invention, and the scope and scope of the present invention are not limited thereto.

### EXAMPLE

### Example 1

Changes in Expression of Genes Related to Lipid Metabolism in Adipocytes by Treatment with Gallocatechin Gallate and Isoquercitrin

The main role of adipocytes is to store excess energy in the form of fat. When excessive energy is accumulated, the amount of fat accumulated in adipocytes increases and adipocytes become enlarged, which appears in the form of body shape changes. Inhibiting the conversion and accumulation of excess nutrients into fat is the most basic solution to obesity.

The present inventors performed the following experiment to determine the effect of a mixture (APGQ) of Gallocatechin Gallate (GCG) and Isoquercitrin (IQC) on the expression of genes regulating lipid metabolism in adipocytes and the mixing ratio of GCG:IQC that can maximize the expression.

3T3-L1 preadipocytes were purchased from ATCC and cultured in Dulbecco's Modified Eagle's Medium (DMEM; Sigma Aldrich) containing 10% bovine calf serum (Gibco) and 1% penicillin/streptomycin (P/S; Sigma Aldrich). 3T3-L1 cells were grown until the culture dish was 100% filled, and then cultured for two more days in the same medium to induce clonal expansion. The differentiation of preadipocytes into adipocytes was performed by treating DMEM containing dexamethasone (1 mM; Sigma Aldrich), insulin (10 mg/ml; Sigma Aldrich), 3-isobutyl-1-methylxanthine (0.5 mM; Sigma Aldrich)+10% fetal bovine serum (FBS; Gibco)+1% P/S for 48 hous and then culturing them with 10% FBS+10 mg/ml insulin+1% P/S DMEM every two days for 10 days to induce differentiation into complete adipocytes.

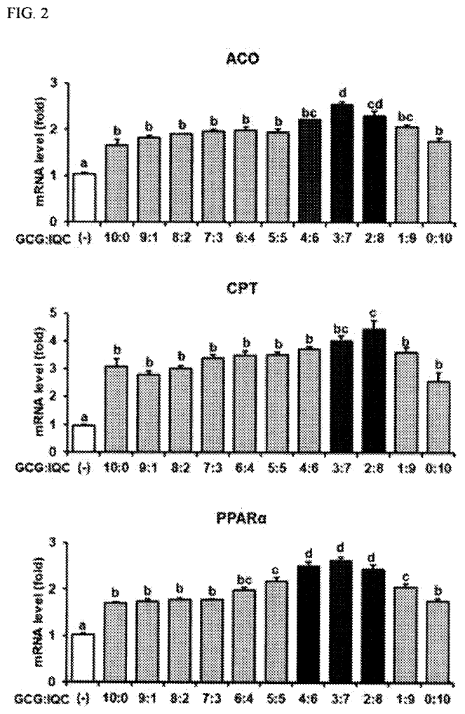

After treating differentiated adipocytes with GCG and IQC at various missing ratios (10.0 to 0:10, GCG+IQC treatment concentration fixed at 100 mg/ml) for 24 hours, the expression of genes related to lipogenesis (FIG. 1) and genes related oxidation of fatty acids (FIG. 2) was measured. For the experiment, the cells were washed twice with phosphate-buffered saline (PBS; Sigma Aldrich), the PBS was removed, and RNA was extracted using the TaKaRa MiniBEST Universal RNA Extraction Kit (Takara Bio). After RNA quantification in the Tecan Infinite M200(Tecan), cDNA was synthesized from the same amount (1 mg) of RNA using the RevertAid Ist-strand cDNA Synthesis Kit (Thermo Fisher Scientific). To observe the expression of target genes, primers were designed using the NCBI primer BLAST (https://www.ncbi.nlm.nih.gov/tools/primer-blast/) service using the coding sequence of the target gene as a template and produced by Bioneer Changes in expression of genes related to fat metabolism were observed using the CFX96 thermocycler (Bio-Rad).

As a result, as shown in FIGS. 1 and 2, although there were differences in composition ratio, it was confirmed that the synergistic effect of the combined treatment of GCG and IQC in controlling lipid metabolism appeared. Although there were slight differences for each lipid metabolism target gene, a significant gene expression regulation effect was observed at a GCG:IQC ratio of 4:6 to 2:8, and in particular, the best effect was observed at 3:7.

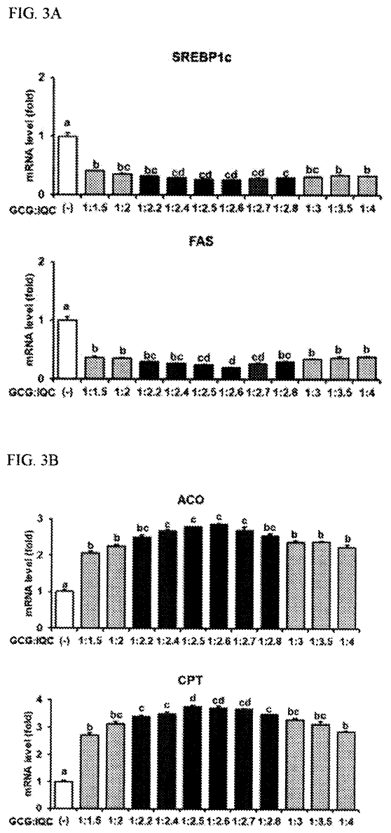

Accordingly, the 4:6 to 2:8 (1:1.5 to 1.4) section of the GCG. IQC ratio was subdivided to reconfirm the expression change patterns of genes related to lipogenesis and oxidation (FIGS. 3A and 3B). As a result, an excellent effect of controlling the expression of genes related to fat metabolism was observed when the GCG:IQC composition ratio was between 1:2 and 1:3. In particular, the best effect was observed when the GCG:IQC ratio was between 1:2.5 and 1:2.6. Therefore, subsequent experiments were conducted with the GCG:IQC ratio of APGQ fixed at 1:2.5.

### Example 2

Activation of Mitochondria and Brown Fat in Adipocytes by Treatment with Gallocatechin Gallate and Isoquercitrin

As confirmed in Example 1, APGQ, which is a mixture of GCG and IQC at a certain ratio, regulated the expression of genes related to the synthesis and decomposition of fatty acid. In the oxidation of fatty acids, neutral fats are broken down to produce free fatty acids, and the free fatty acids move inside the mitochondria and are hydrolyzed to sequentially Generate acetyl-CoA. The generated acetyl-CoA goes through the Kreb cycle and electron transport chain to generate ATP. Mitochondria are present in large quantities in cells of muscle and liver tissue where energy metabolism is active, and adipocytes are known to contain less mitochondria than muscle or liver cells. For fat oxidation to occur smoothly in adipocytes, the amount of mitochondria within adipocytes needs to increase.

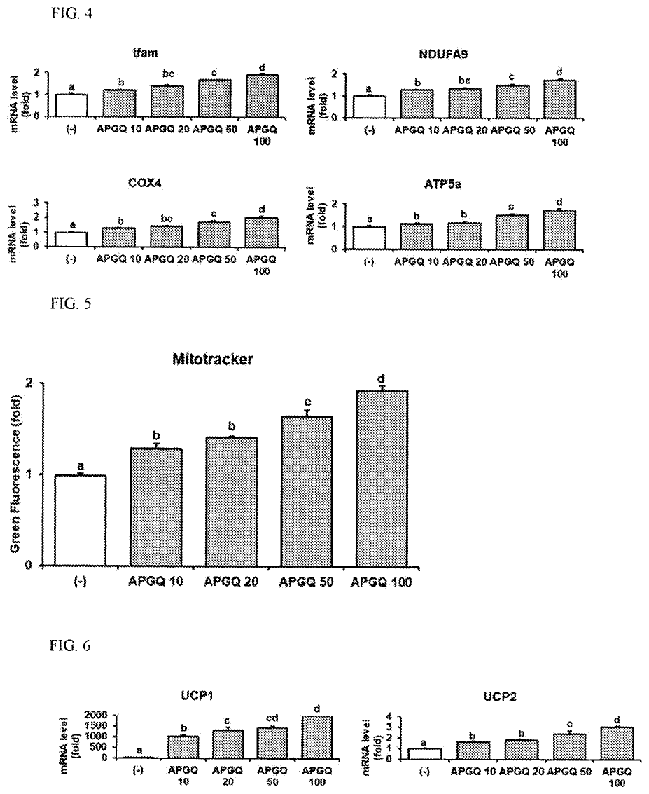

To determine whether APGQ can induce mitochondrial biosynthesis in adipocytes, differentiated adipocytes were treated with APGQ at various concentrations (10 to 100 mg/ml) for 24 hours, RNA was extracted and cDNA was synthesized in the same manner as in Example 1. The expression of mitochondria-related genes was observed through Q-PCR (FIG. 4). Additionally, to determine whether the amount of mitochondria in adipocytes actually increased, differentiated adipocytes were treated with the same concentration of APGQ for 48 hours and then washed with PBS. Afterwards, mitochondria were stained by treating them with Mitotracker™ Green FM (Invitrogen; 20 nM) for 30 minutes, and the fluorescence values were measured and quantified using the TECAN Infinite M200 multiplate reader (ex 490 nm, em 512 nm) (FIG. 5).

As a result, as shown in FIGS. 4 and 5, it was confirmed that when adipocytes were treated with APGQ, the number of mitochondria and the expression of genes related to mitochondria biogenesis (tfam) and electron transport system (NDUFA9, COX4, ATP5a) increase in a concentration-dependent manner. In other words, APGQ can increase energy consumption in adipocytes by inhibiting fatty acid synthesis and promoting fatty acid oxidation while activating mitochondria to facilitate fat metabolism.

In order to synthesize ATP through the mitochondrial electron transport chain, the difference in hydrogen ion concentration inside and outside the mitochondrial inner membrane is very important Sometimes, this difference in concentration of hydrogen ions is used to maintain the temperature necessary for cell survival (thermogenesis) by converting it into heat rather than synthesizing ATP Uncoupling proteins (UCPs) are known to play this role. The tissue specialized for this role is brown fat, which contains may mitochondria, and mainly found in hibernating animals. Interestingly, it has recently been revealed that brown fat also exists in humans, and that the amount is large when young and gradually decreases as the body grows and develops one's temperature control center. The more uncoupling proteins there are, the more ATP is converted to heat rather than produced. Therefore, from the cell's perspective, as UCP increases, more nutrients must be broken down to produce ATP needed to maintain cell function. For this reason, as it was revealed that the distribution and activity of brown fat are closely related to obesity, the activation of brown fat became a new target for obesity treatment. When white fat cells are treated with a b-adrenergic receptor agonist, etc., UCP1 increases along with mitochondria, making them similar to brown fat. These cells are called beige fat cells. What brown fat and beige fat have in common is the presence of large amounts of mitochondria within adipocytes. Accordingly, the expression of UCP was additionally confirmed to determine whether APGQ can induce brown fatization along with an increase in mitochondria in adipocytes (FIG. 6).

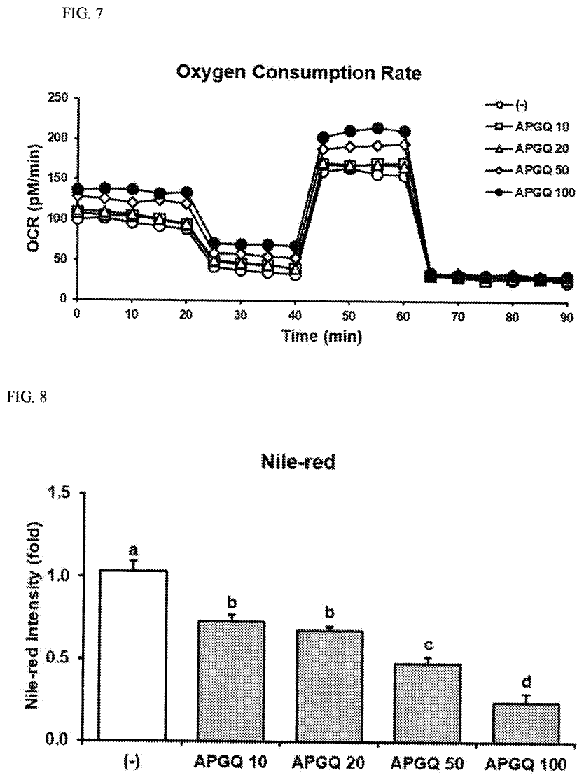

As a result, the expression of UCP1, which exists only in brown fat, increased along with UCP2, which in involved in oxidative stress, indicating that APGQ promotes energy metabolism by inducing brown fat in white fat. In fact, in cells treated with APGQ, cellular oxygen consumption (=oxygen respiration by mitochondria) was found to significantly increase (FIG. 7). This result supports that intracellular energy metabolism was maximized by APGQ treatment.

### Example 3

Inhibitory Effect of Lipid Accumulation in Adipocytes by Treatment with Gallocatechin Gallate and Isoquercitrin

Through Examples 1 and 2, it was confirmed that APGQ inhibits fatty acid synthesis and promotes fat consumption by inducing brown fat in adipocytes. Therefore, the following experiment was performed to determine whether APGQ could actually reduce the amount of fat accumulated in adipocytes through a series of mechanisms.

Differentiated adipocytes were treated with APGQ at various concentrations (10, 20, 50, 100 mg/ml) for 72 hours. Afterwards, the cells were washed twice with PBS and fixed in 10% formaldehyde (Sigma Aldrich) solution for 5 minutes, and then the neutral fat accumulated in the cells was stained using the Nile-red (Sigma Aldrich; 300 nM) solution. The amount of accumulated fat was quantified using the Tecan Infinite M200 Multiplate Reader (ex 495 nm, em 585 nm) (FIG. 8).

As a result, it was confirmed that the amount of neutral fat accumulated in 3T3-L1 adipocytes was reduced through various lipid metabolism control effects upon APGQ treatment.

What is claimed is:

1. A composition for inhibiting lipogenesis; inhibiting fat accumulation; promoting fat breakdown; reducing body fat; and/or preventing, improving or treating obesity or metabolic diseases, comprising an effective amount of Gallocatechin Gallate (GCG) and Isoquercitrin (IQC) in a weight ratio of 1:1 to 1:9.

2. The composition according to claim 1, wherein the composition is a pharmaceutical composition for preventing or treating obesity or metabolic diseases.

3. The composition according to claim 1, wherein the composition is a food composition for inhibiting lipogenesis; inhibiting fat accumulation; promoting fat breakdown; reducing body fat; and/or preventing or improving obesity or metabolic diseases.

4. The composition according to claim 1, wherein the composition is administered to a subject in need of reducing the expression of one or more genes selected from the group consisting of SREBP1c, ACC, and FAS.

5. The composition according to claim 1, wherein the composition is administered to a subject in need of increasing the expression of one or more genes selected from the group consisting of ACO, CPT, PPARα, tfam, NDUFA9, COX4, ATP5a, UCP1, and UCP2.

6. The composition according to claim 1, wherein the composition is administered to a subject in need of increasing mitochondria in adipocytes.

7. The composition according to claim 1, wherein the composition is administered to a subject in need of promoting the production of brown fat.

8. The composition according to claim 1, wherein the daily dosage of the active ingredient of the composition is 5 mg/kg or more.

9. The composition according to claim 1, wherein the daily dosage of the active ingredient of the composition is 10 mg/kg or more.

10. The composition according to claim 1, wherein the composition comprises the gallocatechin gallate and isoquercitrin in a weight ratio of 1:1.5 to 1:4.

11. The composition according to claim 1, wherein the composition comprises the gallocatechin gallate and isoquercitrin in a weight ratio of 1:2.2 to 1:2.8.

12. A method for inhibiting lipogenesis; inhibiting fat accumulation; promoting fat breakdown; reducing body fat; and/or preventing, improving or treating obesity or metabolic diseases, comprising administering an effective amount of the composition of claim 1 to subject in need thereof.

13. The method according to claim 12, wherein the subject is in need of reducing the expression of one or more genes selected from the group consisting of SREBP1c, ACC, and FAS.

14. The method according to claim 12, wherein the subject is in need of increasing the expression of one or more genes selected from the group consisting of ACO, CPT, PPARα, tfam, NDUFA9, COX4, ATP5a, UCP1, and UCP2.

15. The method according to claim 12, wherein the subject is in need of increasing mitochondria in adipocytes.

16. The method according to claim 12, wherein the subject is in need of promoting the production of brown fat.

17. The method according to claim 12, wherein the daily dosage of the active ingredient of the composition is 5 mg/kg or more.

18. The method according to claim 12, wherein the daily dosage of the active ingredient of the composition is 10 mg/kg or more.

19. The method according to claim 12, wherein the composition comprises the gallocatechin gallate and isoquercitrin in a weight ratio of 1:1.5 to 1:4.

20. The method according to claim 12, wherein the composition comprises the gallocatechin gallate and isoquercitrin in a weight ratio of 1:2.2 to 1:2.8.
